(19) United States
(12) Patent Application Publication
(10) Pub. No.: US 2021/0251713 A1
(43) Pub. Date: Aug. 19, 2021

(54) MEDICAL DEVICE PACKAGING SYSTEMS

(71) Applicant: Covidien LP, Mansfield, MA (US)

(72) Inventors: Caroline Atiya, Irvine, CA (US); Thomas E. McNabb, Alta Loma, CA (US)

(21) Appl. No.: 17/248,222

(22) Filed: Jan. 14, 2021

Related U.S. Application Data
(60) Provisional application No. 62/977,688, filed on Feb. 17, 2020.

(51) Int. Cl. A61B 50/33

(52) U.S. Cl. CPC A61B 50/33; A61B 2050/005

(57) ABSTRACT

A medical device package can include a tray defining a recess configured to receive an elongated medical device therein and a lid configured to cover at least a portion of the recess and retain the elongated medical device therein. A retention member or retention aperture formed in the lid permits a clinician to access to a medical device disposed within the recess to releasably retain the elongated medical device with respect to the tray. While releasably retaining the medical device, the clinician to manipulate the device, for example, to sheath or unsheath the device.

### CROSS-REFERENCE TO RELATED APPLICATION

The present application claims the benefit of priority to U.S. Provisional Patent Application No. 62/977,688, filed Feb. 17, 2020, which is incorporated herein by reference in its entirety.

### TECHNICAL FIELD

The present technology relates to systems, devices, and methods for packaging medical devices.

### BACKGROUND

Elongated medical devices, such as catheters, may be packaged in a relatively compact configuration prior to use. For example, a catheter may be stored in a hoop or sheath that is clipped together into a coiled arrangement for shipment and storage prior to use by a clinician during a medical procedure.

### SUMMARY

The present technology is illustrated, for example, according to various aspects described below. Various examples of aspects of the present technology are described as numbered clauses (1, 2, 3, etc.) for convenience. These are provided as examples and do not limit the present technology. It is noted that any of the dependent clauses may be combined in any combination, and placed into a respective independent clause. The other clauses can be presented in a similar manner.

1. A medical device package comprising:

a tray defining a recess configured to receive an elongated medical device therein;

a lid configured to cover at least a portion of the recess and retain the elongated medical device therein; and

a retention aperture formed in the lid, the aperture configured to provide a clinician with access to a medical device disposed within the recess to releasably retain the elongated medical device with respect to the tray.

2. The medical device package of Clause 1, wherein the retention aperture has an area of between about 1 cm² to about 5 cm².

3. The medical device package of any one of the preceding Clauses, wherein the retention aperture is configured to receive a clinician's thumb or finger therethrough.

4. The medical device package of any one of the preceding Clauses, further comprising a medical device disposed within the recess, the elongated medical device comprising:

a catheter having a lumen;

a delivery wire extending through the catheter lumen; and

an interventional element coupled to a distal end portion of the delivery wire, wherein, in a sheathed state, the interventional element is disposed within the catheter lumen, and in an unsheathed state the interventional element is disposed distal to a distal end of the catheter lumen.

5. The medical device package of any one of the preceding Clauses, wherein the interventional element is configured to be transitioned from the unsheathed state to the sheathed state by applying pressure to the catheter through the retention aperture to retain the catheter in position with respect to the tray, and then proximally retracting the interventional element with respect to the catheter by pulling the delivery wire.

6. The medical device package of any one of the preceding Clauses, wherein the interventional element is configured to be transitioned from the sheathed state to the unsheathed state by applying pressure to the catheter through the retention aperture to retain the catheter in position with respect to the tray, and then distally advancing the interventional element with respect to the catheter by pushing the delivery wire.

7. The medical device package of any one of the preceding Clauses, wherein lid is configured to be snap-fit to the tray.

8. The medical device package of any one of the preceding Clauses, wherein the lid is welded to the tray.

9. The medical device package of any one of the preceding Clauses, wherein the recess comprises a lumen of a sheath.

10. The medical device package of any one of the preceding Clauses, wherein the tray comprises clips configured to releasably secure the sheath.

11. The medical device package of any one of the preceding Clauses, further comprising a sheath configured to be disposed within the recess and to receive the elongated medical device therein.

12. The medical device package of any one of the preceding Clauses, wherein a distal end of the sheath is disposed proximal to the retention aperture.

13. The medical device package of any one of the preceding Clauses, wherein the recess extends around an entirety of a perimeter of the tray.

14. The medical device package of any one of the preceding Clauses, wherein the recess defines a spiral channel having a proximal aperture at a radially outermost portion of the tray.

15. The medical device package of any one of the preceding Clauses, wherein the tray and lid each comprise a thermoplastic material.

16. The medical device package of any one of the preceding Clauses, wherein the recess is configured to receive the elongated medical device therein in a coiled state.

17. The medical device package of any one of the preceding Clauses, wherein the coiled state includes a plurality of loops of the elongated medical device.

18. The medical device package of any one of the preceding Clauses, wherein the tray defines a generally spiral planform through the recess.

19. The medical device package of any one of the preceding Clauses, wherein the recess as defined by the tray is continuous with a spiral planform.

20. The medical device package of any one of the preceding Clauses, wherein a distal end portion of the recess comprises an enlarged region configured to receive a distal end portion of the elongated medical device therein.

21. A medical device package comprising:

a tray defining a recess configured to receive an elongated medical device therein;

a lid configured to cover at least a portion of the recess and retain the elongated medical device therein; and

a retention member formed in the lid, the retention member configured to be releasably depressed into contact with a medical device disposed within the recess.

22. The medical device package of any one of the preceding Clauses, wherein the retention member comprises a button.

23. The medical device package of any one of the preceding Clauses, wherein the retention member has an area of between about 1 cm² to about 5 cm².

24. The medical device package of any one of the preceding Clauses, wherein the retention member is configured to receive a clinician's thumb or finger thereon.

25. The medical device package of any one of the preceding Clauses, further comprising a medical device disposed within the recess, the elongated medical device comprising:

a catheter having a lumen;

a delivery wire extending through the catheter lumen; and

an interventional element coupled to a distal end portion of the delivery wire, wherein, in a sheathed state, the interventional element is disposed within the catheter lumen, and in an unsheathed state the interventional element is disposed distal to a distal end of the catheter lumen.

26. The medical device package of any one of the preceding Clauses, wherein the interventional element is configured to be transitioned from the unsheathed state to the sheathed state by depressing the retention member to retain the catheter in position with respect to the tray, and then proximally retracting the interventional element with respect to the catheter by pulling the delivery wire.

27. The medical device package of any one of the preceding Clauses, wherein the interventional element is configured to be transitioned from the sheathed state to the unsheathed state by depressing the retention member to retain the catheter in position with respect to the tray, and then distally advancing the interventional element with respect to the catheter by pushing the delivery wire.

28. The medical device package of any one of the preceding Clauses, wherein lid is configured to be snap-fit to the tray.

29. The medical device package of any one of the preceding Clauses, wherein the lid is welded to the tray.

30. The medical device package of any one of the preceding Clauses, wherein the recess comprises a lumen of a sheath.

31. The medical device package of any one of the preceding Clauses, wherein the tray comprises clips configured to releasably secure the sheath.

32. The medical device package of any one of the preceding Clauses, further comprising a sheath configured to be disposed within the recess and to receive the elongated medical device therein.

33. The medical device package of any one of the preceding Clauses, wherein a distal end of the sheath is disposed proximal to the retention member.

34. The medical device package of any one of the preceding Clauses, wherein the recess extends around an entirety of a perimeter of the tray.

35. The medical device package of any one of the preceding Clauses, wherein the recess defines a spiral channel having a proximal aperture at a radially outermost portion of the tray.

36. The medical device package of any one of the preceding Clauses, wherein the tray and lid each comprise a thermoplastic material.

37. The medical device package of any one of the preceding Clauses, wherein the recess is configured to receive the elongated medical device therein in a coiled state.

38. The medical device package of any one of the preceding Clauses, wherein the coiled state includes a plurality of loops of the elongated medical device.

39. The medical device package of any one of the preceding Clauses, wherein the tray defines a generally spiral planform through the recess.

40. The medical device package of any one of the preceding Clauses, wherein the recess as defined by the tray is continuous with a spiral planform.

41. The medical device package of any one of the preceding Clauses, wherein a distal end portion of the recess comprises an enlarged region configured to receive a distal end portion of the elongated medical device therein.

42. A method comprising:

providing a medical device package assembly comprising:
a tray defining a recess;
an elongated medical device disposed within the recess, the elongated medical device comprising a catheter and an interventional element;
a lid configured to cover at least a portion of the recess; and
a retention aperture formed in the lid;
   contacting the catheter through the retention aperture;
   manipulating the interventional element while contacting the catheter, thereby moving the interventional element proximally or distally with respect to the catheter.

43. The method of any one of the preceding Clauses, wherein manipulating the interventional element comprises distally advancing or proximally retracting a delivery member coupled to the interventional element.

44. The method of any one of the preceding Clauses, wherein manipulating the interventional element comprises distally advancing the interventional element beyond a distal end of the catheter.

45. The method of any one of the preceding Clauses, wherein manipulating the interventional element comprises proximally retracting the interventional element from a first position distal to a distal end of the catheter to a second position proximal to the distal end of the catheter.

46. The method of any one of the preceding Clauses, wherein the interventional element is self-expanding.

47. The method of any one of the preceding Clauses, wherein contacting the catheter comprises pressing a thumb or finger onto the catheter through the retention aperture.

48. A method comprising:

providing a medical device package assembly comprising:
a tray defining a recess;
an elongated medical device disposed within the recess, the elongated medical device comprising a catheter and an interventional element;
a lid configured to cover at least a portion of the recess; and
a retention member formed in the lid;
   depressing the retention member into contact with the catheter;
   manipulating the interventional element while depressing the retention member into contact with the catheter, thereby moving the interventional element proximally or distally with respect to the catheter.

49. The method of any one of the preceding Clauses, wherein manipulating the interventional element comprises distally advancing or proximally retracting a delivery member coupled to the interventional element.

50. The method of any one of the preceding Clauses, wherein manipulating the interventional element comprises distally advancing the interventional element beyond a distal end of the catheter.

51. The method of any one of the preceding Clauses, wherein manipulating the interventional element comprises proximally retracting the interventional element from a first position distal to a distal end of the catheter to a second position proximal to the distal end of the catheter.

52. The method of any one of the preceding Clauses, wherein the interventional element is self-expanding.

53. The method of any one of the preceding Clauses, wherein contacting the catheter comprises pressing a thumb or finger onto the retention member.

Additional features and advantages of the present technology are described below, and in part will be apparent from the description, or may be learned by practice of the present technology. The advantages of the present technology will be realized and attained by the structure particularly pointed out in the written description and claims hereof as well as the appended drawings.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1A depicts a top perspective view of a medical device package in accordance with embodiments of the present technology.

FIG. 1B depicts a perspective exploded view of the medical device package of FIG. 1A.

FIG. 1C depicts a top view of a lid portion of the medical device package of FIGS. 1A and 1B.

FIG. 1D depicts a top view of tray and sheath portions of the medical device package of FIGS. 1A and 1B.

FIGS. 2A and 2B illustrate top perspective and top views, respectively, of another example tray portion of a medical device package in accordance with embodiments of the present technology.

FIGS. 3A and 3B depict side schematic views of a medical device in an unsheathed and sheathed state, respectively, for positioning within a medical device package in accordance with embodiments of the present technology.

FIG. 4A depicts a top perspective view of another medical device package in accordance with embodiments of the present technology.

FIG. 4B depicts a perspective exploded view of the medical device package of FIG. 4A.

FIG. 4C depicts a top view of a lid portion of the medical device package of FIGS. 4A and 4B.

FIG. 4D depicts a top view of tray and sheath portions of the medical device package of FIGS. 4A and 4B.

FIG. 5A depicts a top perspective view of another medical device package in accordance with embodiments of the present technology.

FIG. 5B depicts a perspective exploded view of the medical device package of FIG. 5A.

FIG. 5C depicts a top view of a lid portion of the medical device package of FIGS. 5A and 5B.

FIG. 5D shows a top view of tray and sheath portions of the medical device package of FIGS. 5A and 5B.

### DETAILED DESCRIPTION

Elongated medical device assemblies may be packaged for shipment and storage after manufacture and before being used in a medical procedure. An elongated medical device assembly may be packaged in a configuration other than a linear configuration (e.g., where the device assembly may be held in a straight configuration) in order to provide for more efficient storage of the elongated medical device assembly (e.g., by storing the medical device assembly in a package having a footprint with at least one smaller dimension than the linear configuration of the medical device assembly). For example, the medical device may be stored in a package having a smaller length than the total length of the medical device assembly, measured along a longitudinal axis of the medical device assembly. In addition, storing the elongated medical device assembly in a non-linear configuration may help reduce shipping costs, provide for easier manipulation of the medical device package, or any combination thereof.

The medical device package may also help isolate the elongated medical device assembly from the surrounding environment, help protect the structural integrity of the medical device, help prevent contamination of the elongated medical device assembly during shipping and/or storage, or both help protect the structural integrity of the medical device and help prevent contamination of the medical device.

Some medical device packages may include an outer sheath (e.g., relatively rigid polymeric tubing) that receives the elongated medical device therein. The sheath of the medical device packaging may be clipped to itself or otherwise fused/held together in a coiled shape, such that the elongated medical device assembly is received within the sheath while the sheath is in the coiled shape. Such a medical device packaging sheath may be referred to as a “snail pack.” A clinician may remove the elongated medical device assembly from the snail pack by longitudinally pulling the elongated medical device assembly out of one end of the sheath of the snail pack (e.g., pulling the elongated medical device assembly along a longitudinal axis of the elongated medical device assembly).

In some embodiments, an elongated medical device assembly includes a catheter, a delivery wire extending through a lumen of the catheter, and a medical device coupled to a distal end of the delivery wire. The medical device may be, for example, an intrasaccular implant, a thrombectomy device, another intravascular device, or any other suitable medical device. In some instances, the medical device can assume a constrained, low-profile configuration while sheathed (e.g., disposed within the catheter of the lumen), and can assume an unconstrained, radially expanded configuration when unsheathed (e.g., positioned outside the catheter).

In conventional packaging systems, it can be difficult to manipulate the medical device assembly while it is disposed within the package. For example, in some medical device assemblies, a medical device (e.g., an implant or interventional element) coupled to a delivery wire can be slidably advanced or retracted relative to a surrounding catheter. In conventional packaging approaches, such medical devices remain in a constrained, low-profile configuration within the catheter while the medical device assembly is disposed within the outer sheath of the package. This allows the medical device assembly to be pulled proximally through the lumen of the outer sheath for use by a clinician, while protecting the device from damage during retraction through the outer sheath. However, this low-profile, constrained configuration can be damaging to certain medical devices. For example, braided implants having very small wire diameters can be damaged if the implant remains in the low-profile, constrained configuration for extended periods. Among other problems, the implant may be plastically deformed and/or may lose the ability to self-expand to the desired shape upon deployment within the body. Accordingly, it can be beneficial to package such medical device assemblies in a manner that permits the medical device to remain its expanded and unconstrained state while within the package. However, to facilitate removal of the medical device assembly from the package (e.g., pulling the medical device assembly through the outer sheath), the medical device may need to be transitioned into the low-profile, constrained configuration within the catheter before the medical device assembly is pulled through the surrounding outer sheath. Accordingly, there is a need to provide packaging systems for medical device assemblies that permit a medical device assembly to be manipulated while remaining within the package. For example, while an elongated medical device assembly is disposed within a package (e.g., extending through the lumen of the outer sheath), the medical device can be transitioned between a low-profile, constrained configuration within the lumen of the catheter, and an expanded, unconstrained configuration positioned outside the lumen of the catheter.

As described in more detail below, embodiments of the present technology provide a medical device package having a retention aperture or retention member that facilitates manipulation of a medical device assembly while the assembly remains within the package. For example, a retention aperture can provide a window through which a clinician may press down on a catheter of the medical device assembly to hold the catheter in place. While holding the catheter securely, the delivery wire of the medical device assembly can be distally advanced or proximally retracted to move the delivery wire and the medical device relative to the surrounding catheter. (As described in more detail below, in some embodiments a retention member can be used instead of an aperture, in which case a portion of the package lid can be depressed into contact with the catheter or other portion of the medical device assembly). By moving the medical device distally or proximally relative to the surrounding catheter, the medical device can be transitioned between its low-profile, constrained configuration within the lumen of the catheter and the expanded, unconstrained configuration outside the lumen of the catheter.

In some embodiments, the medical device assembly can be inserted into the package while the medical device is in the low-profile, constrained configuration (e.g., the medical device can be disposed within the lumen of the catheter). This can protect the medical device from damage while being positioned within the package (e.g., while being slidably advanced through a lumen of the outer sheath). Once the medical device assembly has been placed within the package, a clinician can utilize the retention aperture to secure the catheter while distally advancing the delivery wire and the medical device beyond the catheter, such that the medical device assumes the expanded, unconstrained state. The medical device assembly may remain in this configuration within the package until it is ready for use by a clinician. Once the medical device assembly is ready to be used, a clinician may again utilize the retention aperture to secure the catheter in place while resheathing the device, e.g., by proximally retracting the delivery wire and medical device relative to the surrounding catheter in order to urge the medical device into the lumen of the catheter, where it assumes the low-profile, constrained configuration. Once in this position, the medical device assembly can be removed from the package (e.g., by being pulled through the lumen of the outer sheath). As a result of this arrangement, the medical device can remain in the expanded, unconstrained configuration during the majority of its time within the package, with the device only assuming the low-profile, constrained configuration during insertion into the package and removal from the package. Accordingly, medical devices that are susceptible to deformation or other damage can benefit from the packaging systems disclosed herein, which permit manipulation of medical device assemblies while disposed within the package.

In various embodiments the package can take the form of a tray portion removably coupled to a lid portion. The tray portion and/or the lid portion may define one or more channels, recesses, or grooves configured to receive the elongated medical device assembly therein, for example in a coiled configuration. In some embodiments, the medical device assembly may be disposed within an outer sheath which is disposed within the channel or recess. In other embodiments, the medical device assembly may be disposed within the channel or recess without being positioned within an outer sheath.

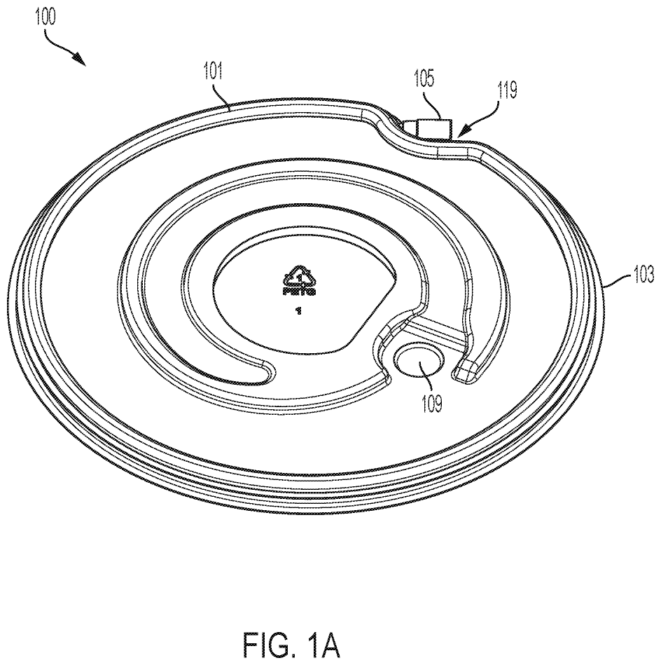

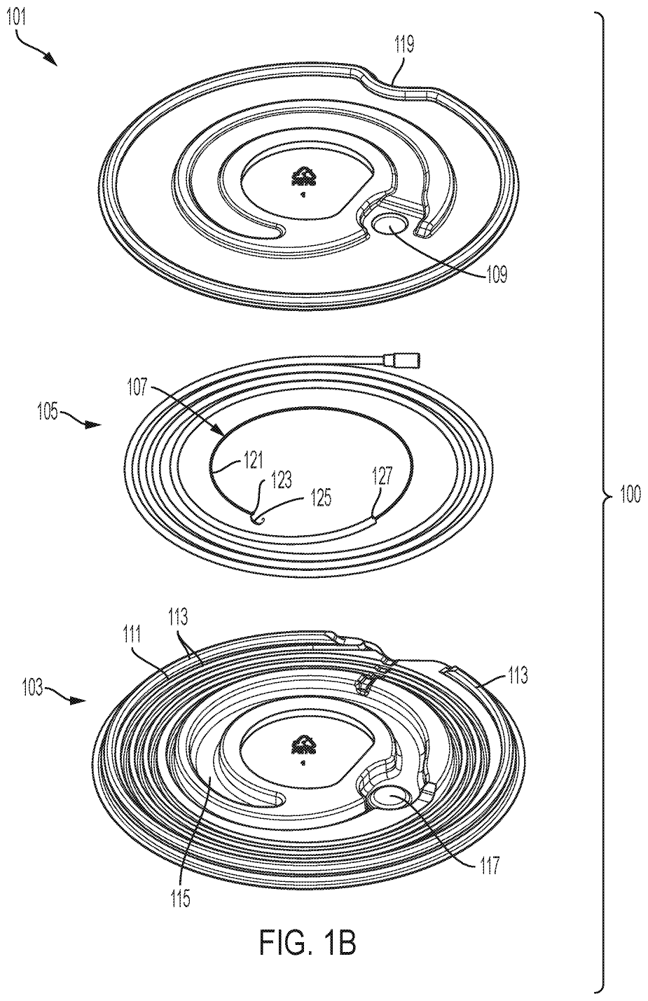

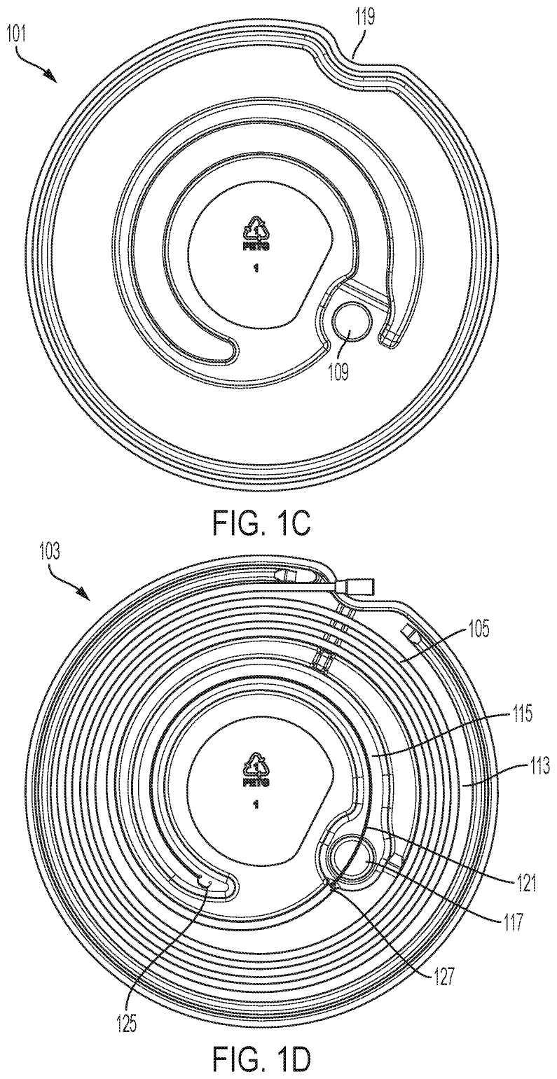

In some cases, it can be useful to hydrate a medical device while it remains within the package. For example, a clinician may fully hydrate the elongated medical device, e.g., to activate a hydrophilic coating of the medical device, while the elongated medical device is still within the tray. As an example, the clinician may remove the tray in which the elongated medical device is housed from a sealed pouch and place the tray within a hydration container filled with a saline solution or another hydrating fluid. In some examples, the tray may include drain holes configured to drain a liquid within the tray, providing further time savings as such a medical device package could be removed from a hydration container and placed on a surface to drain while the clinician attends to other activities. In some embodiments of the present technology, the package can be configured to facilitate hydration of the medical device. For example, hydrating fluid can be disposed within the channel or recess in the tray portion, such that the medical device is at least partially covered with the hydrating fluid while disposed within the channel or recess. In some examples, the entire package can be at least partially submerged in hydrating fluid (or hydrating fluid can be applied over at least a portion of the package), thereby covering at least a portion of the medical device assembly therein with the hydrating fluid. FIGS. 1A depicts a top perspective view of a medical device package 100 including a lid portion 101 removably mated with a tray portion 103. An outer sheath 105 or hoop that is disposed between the lid portion 101 includes an elongated medical device assembly 107 therein. The lid portion 101 includes a retention member 109 that can facilitate manipulation of the medical device assembly 107 within the package 100. FIG. 1B depicts a perspective exploded view of the medical device package 100. FIG. 1C depicts a top view of the lid portion 101, and FIG. 1D illustrates a top view of the tray portion 103 with the outer sheath 105 disposed therein.

Referring to FIGS. 1A-1D together, the package 100 is configured to receive and house the elongated medical device assembly 107 in a coiled state (e.g., in a coiled shape as shown in FIG. 1B) in a channel or recess 111 within the tray portion 103. The recess 111 can be defined by ridges or sidewalls 113 extending along either side of the recess 111 and forming a generally spiral shape as seen from above (e.g., a spiral planform). At a radially innermost portion of the recess 111 (e.g., at a distal end portion of the recess 111) is an expanded region 115 in which the sidewalls 113 are further apart and the recess 111 has a wider dimension. This expanded region 115 can be configured to receive the distal end portion of the medical device assembly 107 therein. The lid portion 101 is configured to extend over and cover at least a portion of recess 111 of the tray portion 103, which may help retain medical device assembly 107 within the recess 111.

The tray portion 103 can also include an engagement surface 117 configured to be aligned with the retention member 109 of the lid portion 101 when tray portion 103 and the lid portion 101 are mated together. The engagement surface 117 can be disposed within the recess 111, for example at a proximal portion of the expanded region 115. In some embodiments, the engagement surface 117 includes a raised surface relative to adjacent portions of the recess 111, for example having a circular or other suitable shape that protrudes upward from the lower surface of the recess 111. In some embodiments, the retention member 109 can take the form of a button, protrusion, indentation, recess, or other feature configured to be depressed with respect to the lid portion and protrude towards the tray portion 103. In some embodiments, the retention member 109 can have a surface area of between about 1 cm² to about 5 cm². In some embodiments, the retention member 109 can be configured to flex or deform when pressed by a clinician so as to come into contact with the engagement surface 117. For example, when the lid portion 101 is mated to the tray portion 103, a clinician may press down on the retention member 109 such that it comes moves towards the engagement surface 117, for example coming into direct contact with the engagement surface 117. In operation, the medical device assembly 107 can be positioned between the retention member 109 and the engagement surface 117, such that depressing the retention member 109 frictionally engages the medical device assembly 107 therebetween.

When mated together, the tray portion 103 and the lid portion 101 can define a proximal mouth 119 in fluid communication with the recess 111. In some embodiments, the outer sheath 105 can be loaded into the package 100 by slidably inserting the outer sheath 105 into the recess 111 through the proximal mouth 119.

As noted previously, the elongated medical device assembly 107 can be disposed within the recess 111 of the tray portion 103. The medical device assembly 107 can be any suitable medical device, such as, but not limited to, a catheter, a guidewire, an implant delivery system such as a intrasaccular implant delivery device, an instrument such as a thrombectomy device located at the end of an elongated member such as a wire, hypotube, a combination of wire and hypotube, an electrically conductive lead, an optical fiber, a catheter-deliverable heart valve, or another elongated vascular device. In the example shown in FIG. 1B, the medical device assembly 107 includes a catheter 121 and a delivery wire 123 extending through a lumen of the catheter 121. A medical device 125 is coupled to the distal end of the delivery wire 123. The medical device assembly 107 can be slidably moveable relative to the outer sheath 105. Additionally, the delivery wire 123 and the medical device 125 can, together, be slidably moveable relative to the surrounding catheter 121. For example, the delivery wire 123 can be distally advanced relative to the catheter 121 until the medical device 125 is disposed outside the lumen of the catheter 121. In the case of a self-expanding medical device 125, the medical device 125 can assume an unconstrained, expanded configuration when disposed beyond the lumen of the catheter 121. When the delivery wire 123 is proximally retracted relative to the catheter 121 (or the catheter 121 is distally advanced relative to the delivery wire 123), the medical device 125 can be urged back into the lumen of the catheter 121, where it assumes a low-profile, constrained configuration.

For certain medical devices, it can be beneficial to minimize the amount of time the medical device 125 is stored in the low-profile, constrained configuration within the lumen of the catheter 121. For example, for certain woven mesh implants having small diameter wires, prolonged periods in a constrained configuration may impart an undesirable shape set to the implant, thereby reducing efficacy once deployed within a patient. Accordingly, while the medical device assembly 107 is stored within the package 100, it can be beneficial to provide a mechanism for transitioning the medical device 125 between a constrained, low-profile configuration (e.g., within the lumen of the catheter 121) and an unconstrained, expanded configuration (e.g., outside the lumen of the catheter 121).

In some embodiments, the retention member 109 permits a clinician to releasably secure the catheter 121 while manipulating the delivery wire 123 to move the medical device 125 between a first position within the lumen of the catheter 121 and a second position outside the lumen of the catheter 121. For example, as best seen in FIG. 1D, a distal end 127 of the outer sheath 105 can be disposed proximal to the engagement surface 117. The medical device assembly 107 extends distally beyond the distal end 127 of the outer sheath 105 and into the enlarged region 115 of the recess 111. The medical device assembly 107 therefore extends over the engagement surface 117. When the lid portion 101 is mated with the tray portion 103, a portion of the medical device assembly 107 is disposed between the engagement surface 117 of the tray portion 103 and the retention member 109 of the lid portion 101. When a clinician depresses the retention member 109, it is urged into contact with the medical device assembly 107, particularly holding the catheter 121 into frictional engagement between the retention member 109 and the engagement surface 117. While holding the catheter 121 in position, the clinician may then manipulate the position of the medical device 125 with respect to the catheter 121 (e.g., by pushing or pulling a proximal portion of the delivery wire 123). Once the clinician has moved the medical device 125 into its desired position, the clinician can release the retention member 109, after which the catheter 121 can be slidably moved with respect to the outer sheath 105.

In some examples, lid portion 101 and tray portion 103 may be manufactured as a single unitary structure. In other examples, lid portion 101 may be manufactured as a separate component from tray portion 103. In such examples, tray portion 103 may be configured to receive lid portion 101 to define the proximal mouth 119 through which the outer sheath 105 can be slidably inserted. For example, the lid portion 101 and/or the tray portion 103 can be configured to assume a generally planar, circular, partially circular, or semi-circular shape. Whether lid portion 101 and tray portion 103 are manufactured as a single unitary structure or are manufactured as separate components, lid portion 101 and tray portion 103 may be configured to be either moveable relative to each other or fixed relative to each other. For example, lid portion 101 may be attached to tray portion 103 such that lid portion 101 is hinged as in a clamshell configuration. In some examples, lid portion 101 and tray portion 103 may be configured to be moveable relative to each other when medical device package 100 has received elongated medical device assembly 107 (e.g., rather than lid portion 101 and tray portion 103 only being “moveable” and that lid portion 101 and tray portion 103 are assembled together in preparation of receiving elongated medical device assembly 107).

In examples in which lid portion 101 is configured to be a separate manufactured component from tray portion 103, lid portion 101 may be configured to attach to tray portion 103 to prevent lid portion 101 from separating from tray portion 103 during use of medical device package 10. For example, a channel of tray portion 103 may be configured to receive a corresponding ridge of the lid portion 101, for example via an interference fit. In these examples, tray portion 103 may be configured to define the location of lid portion 101 through the specific configuration of the channel.

In other examples, lid portion 101 may be configured to attach to tray portion 103 in a pseudo-permanent fashion, such that lid portion 101 and tray portion 103 may not be separated without physically damaging medical device package 100. For example, lid portion 101 may be welded, fused, glued, or pressed into tray portion 103 such that lid portion 101 is configured to not separate from tray portion 103.

The tray portion 103 and lid portion 101 may be formed from any suitable material, such as but not limited to, polyethylene terephthalate glycol-modified (PETG), Tritan, or other suitable polymers or materials. The tray portion 103 and lid portion 101 can be formed using any suitable technique, for example thermoforming, injection molding, 3D printing, etc. In some examples, medical device package 100 may be configured to withstand sterilization procedures for elongated medical device assembly 107. For example, medical device package 100 may be formed from a material configured to withstand being exposed to a predetermined sterilization temperature for a predetermined period of time. For example, the predetermined sterilization temperature may be about 50° C. In some examples, the predetermined period of time may be about 1 second, about 10 seconds, about 1 minute, about 30 minutes, about 1 hour, about 2 hours, or more than 2 hours. In some examples, in addition to, or instead of, exposing medical device package 100 and received elongated medical device assembly 107 to the predetermined sterilization temperature, medical device package 100 may be configured to withstand other sterilization techniques. For example, medical device package 100 may be configured to withstand microwave, steam, ethylene oxide or ozone sterilization, or other sterilization techniques. In some examples, the sterilization may be performed after sealing tray portion 103, lid portion 101, and elongated medical device assembly 107 in a pouch of medical device package 100. In other examples, the sterilization may be performed before sealing elongated medical device assembly 107 in a pouch of medical device package 100.

In some examples, all or part of medical device package 100 may be manufactured from an opaque or transparent material. Configuring medical device package 100 to be at least partially transparent may provide one or more advantages. For example, if elongated medical device assembly 107 may be inspected when elongated medical device assembly 107 is in a coiled state within recess 111, elongated medical device assembly 107 may require less handling prior to use in a medical procedure.

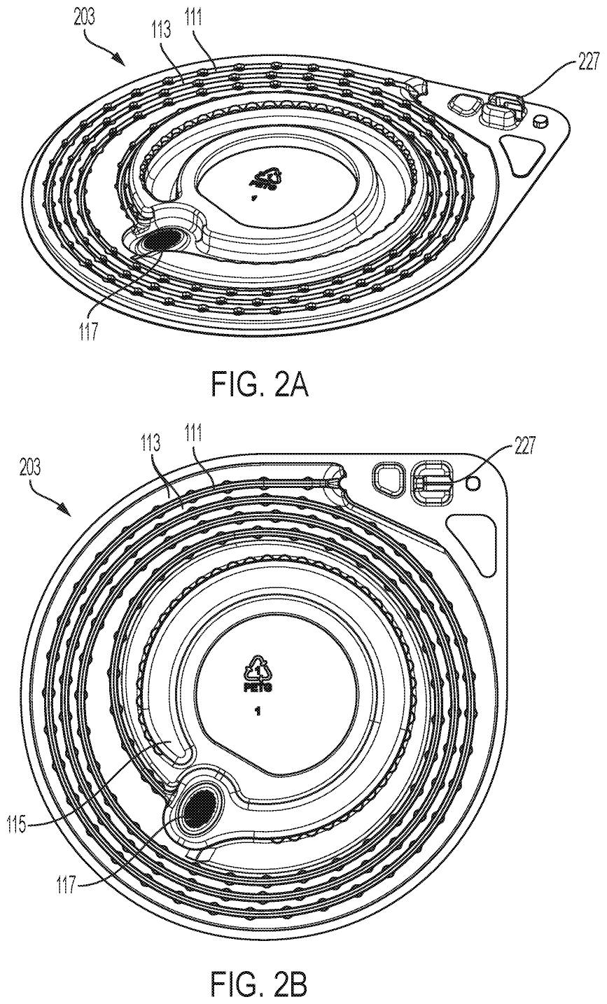

FIGS. 2A and 2B illustrate top perspective and top views, respectively, of another example tray portion 203 for use in a medical device package. For example, the tray portion 203 can be used with the medical device assembly 107 and the lid portion 101 described above with respect to FIGS. 1A-1D. The tray portion 203 can be similar to tray portion 103 described previously, except that the tray portion 203 includes a recess 111 defined by sidewalls 113 that is configured to receive a medical device assembly 107 directly therein, without the presence of an outer sheath 105. For example, the recess 111 defined by the tray portion 203 can be narrower than that of the tray portion 103 shown in FIG. 1B. In operation, a medical device assembly 107 can be loaded into the recess 111 by being slidably advanced through the recess 111 until a distal portion of the medical device assembly 107 extends into the enlarged region 115. Retention and manipulation of the medical device assembly 107 can be achieved via the engagement surface 117 and a retention member of a lid portion 101 similar to that described above.

In the illustrated example, the tray portion 203 additionally includes a receptacle 227 configured to releasably receive a proximal portion of the medical device assembly 107. For example, the depicted tray portion 203 is configured to receive a proximal hub, e.g., via a snap fit into the receptacle 2. The retention system may include indentations and/or protrusions shaped to securely receive a hub or other portion of a medical device assembly 107 therein. In some examples, receptacle 227 may include features defined by tray portion 203, for example a physical depression or protrusion within a surface of tray portion 203 that is configured to receive a proximal hub via an interference fit or other technique.

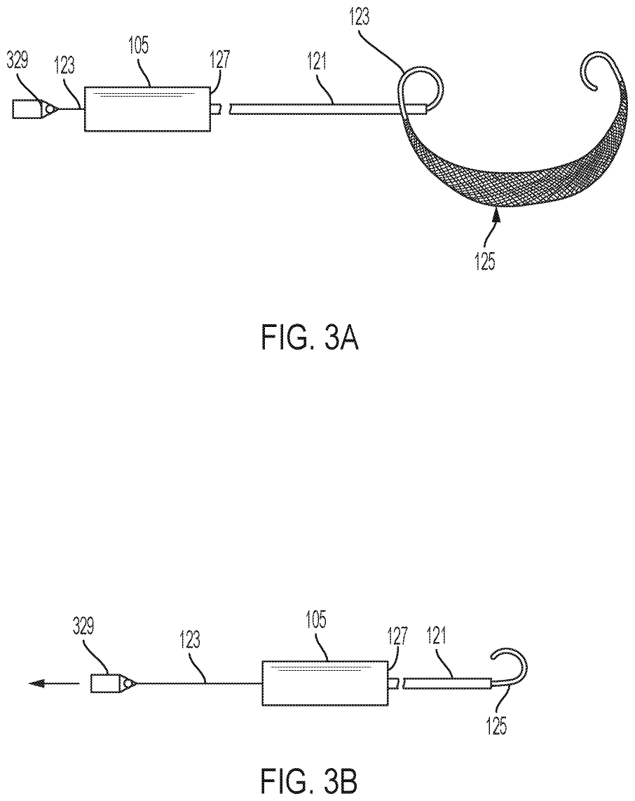

FIGS. 3A and 3B depict side schematic views of an example medical device 125 in an unsheathed and sheathed state, respectively. As seen in FIG. 3A, the medical device 125 can take the form of an implant coupled to a distal end of a delivery wire 123. The delivery wire 123 extends through a lumen of the catheter 121, which in turn extends through a lumen of the outer sheath 105. A proximal end 329 of the delivery wire 123 can include a hub or other component to facilitate manipulation of the delivery wire 123. In the configuration shown in FIG. 3A, the medical device 125 has been distally advanced beyond the catheter 121, such that the medical device 125 assumes an unconstrained, expanded state.

As noted elsewhere herein, in some embodiments it may be desirable to package a medical device 125 such that the device 125 remains in the unconstrained, expanded state until the clinician is ready to remove the device 125 from the package. At that stage, as depicted in FIG. 3B, the clinician can resheath the medical device 125 by proximally retracting the delivery wire 123 (e.g., by pulling proximally on the proximal end 329). As the medical device 125 is pulled proximally relative to the catheter 121, the medical device 125 can be urged into its constrained, low-profile configuration until the device 125 is disposed partially or completely within the lumen of the catheter 121. Once in this state, the catheter 121 can be retracted proximally through the outer sheath 105 and the medical device assembly 107 can be prepared for use.

The medical device 125 illustrated in FIG. 3A is one example of a suitable medical device that may benefit from the packages disclosed herein. For example, the medical device 125 can be an implant made of small-diameter braided filaments and configured to be placed across the neck of an aneurysm. Particular details of such exemplary medical devices can be found in U.S. application Ser. No. 16/718,171, filed Dec. 17, 2019 and titled “Devices, Systems, and Methods for the Treatment of Vascular Defects,” which is hereby incorporated by reference in its entirety. In various examples, any suitable medical device may be used in conjunction with the packages disclosed herein.

Although several examples described herein refer to a “delivery wire,” in some embodiments the medical device may be coupled to an elongate manipulation member other than a wire, for example a hypotube, a multi-component elongate manipulation member, or any other suitable elongate structure configured to extend through a catheter lumen and couple either removably or non-removably to a medical device.

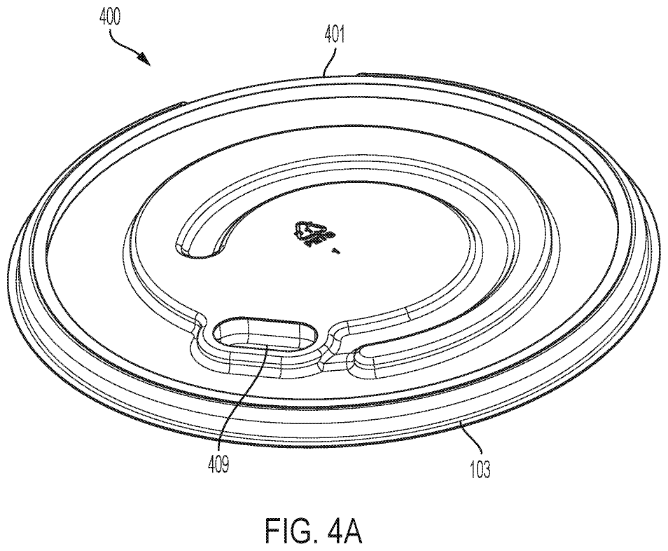

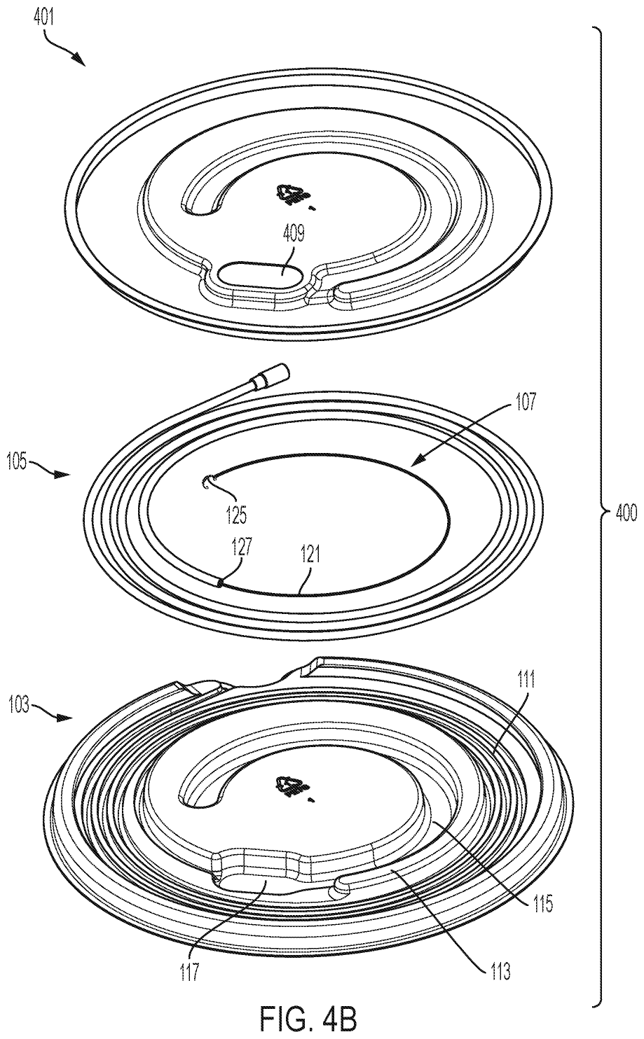

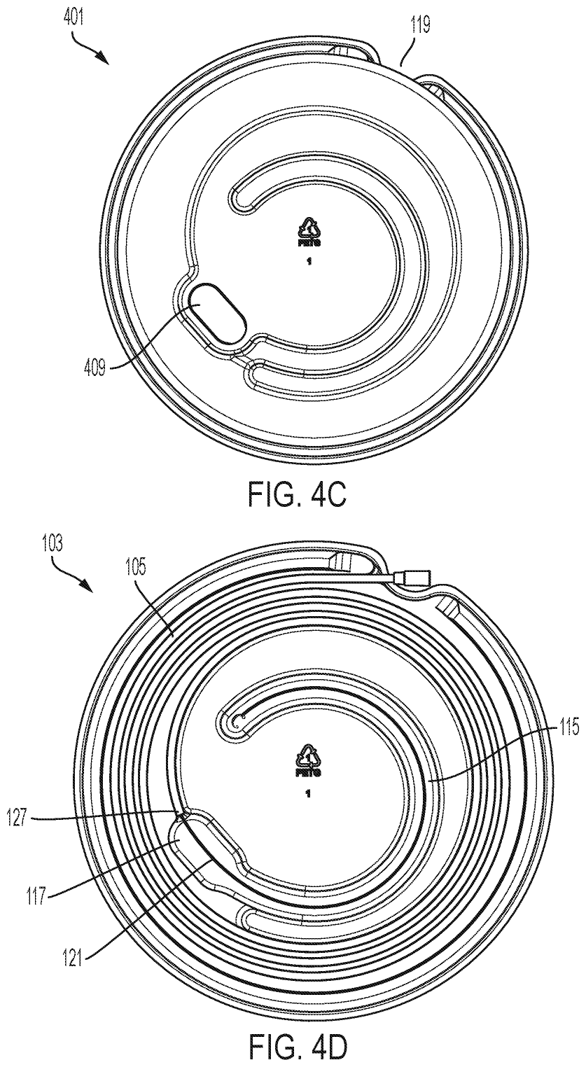

FIG. 4A depicts a top perspective view of another medical device package 400 including a lid portion 401 mated to a tray portion 403 and enclosing an outer sheath 105 therebetween 105. FIG. 4B depicts a perspective exploded view of the medical device package 400, FIG. 4C depicts a top view of the lid portion 401, and FIG. 4D illustrates a top view of the tray portion 403 with the outer sheath 405 and the medical device assembly 407 disposed thereon. In various embodiments, the package 400 can include some or all of the features of the medical device packages 100 or 500 described elsewhere herein.

Referring to FIGS. 4A-4D together, the outer sheath 105, medical device assembly 107, and tray portion 103 can be similar to those described above with respect to FIGS. 1A-1D. However, the lid portion 401 can include a retention aperture 409 formed therein. The retention aperture 409 can take the form of a window, void, or opening in the body of the lid portion 401 that is configured to permit a clinician to insert a finger or instrument therethrough. In some embodiments, the retention aperture 409 can define an area of between about 1 cm² to about 5 cm². When the lid portion 401 is mated with the tray portion 403, the retention aperture 409 can be aligned with the engagement surface 117 such that a clinician can touch the engagement surface 117 through the retention aperture 409.

In operation, when the outer sheath 105 and medical device assembly 107 are disposed within the recess 111 of the tray portion 103, the distal end of the outer sheath 105 terminates proximal to the engagement surface 117 such that the catheter 121 of the medical device assembly 107 extends over the engagement surface 117 and is visible through the retention aperture 409. To manipulate the medical device 125 with respect to the catheter 121, the clinician can use a finger or instrument inserted through the retention aperture 409 to press the catheter 121 against the engagement surface 117 to retain the catheter 121 in place. While holding the catheter 121 in this position, the clinician can manipulate the delivery wire 123 (e.g., by pulling or pushing on a proximal end portion of the delivery wire 123), thereby distally advancing or proximally retracting the medical device 125 relative to the catheter 121. As described elsewhere herein, this can enable the medical device 125 to be stored for extended periods in an unconstrained state, outside of the catheter 121, and to be moved into a constrained state within the catheter 121 when the device 125 is being prepared for use.

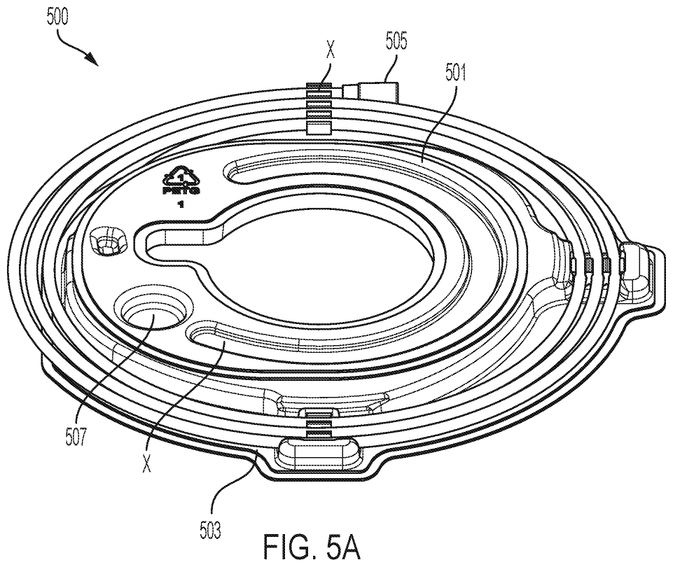

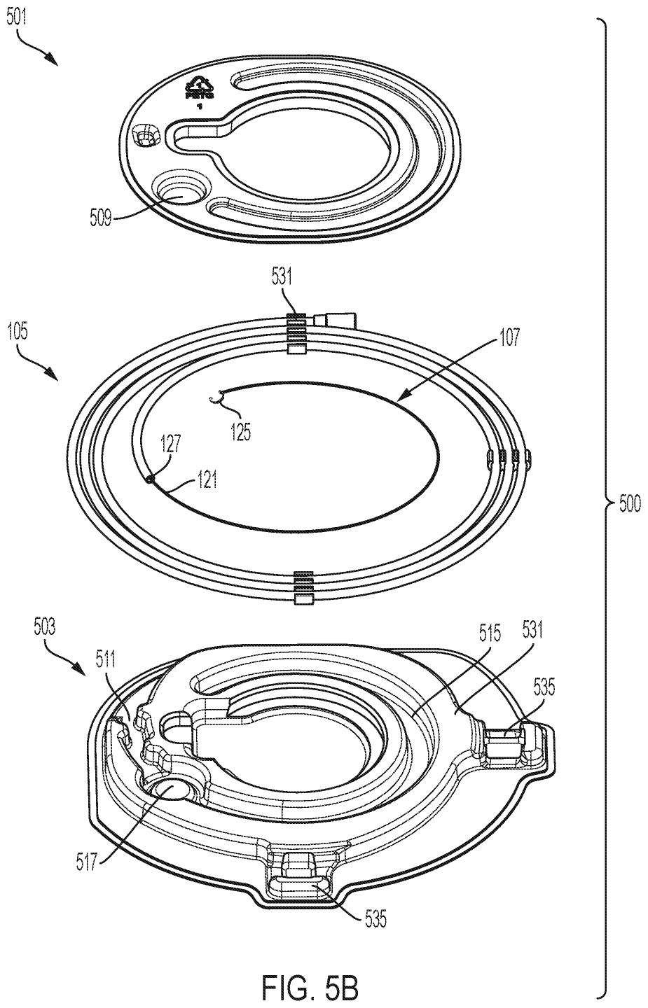

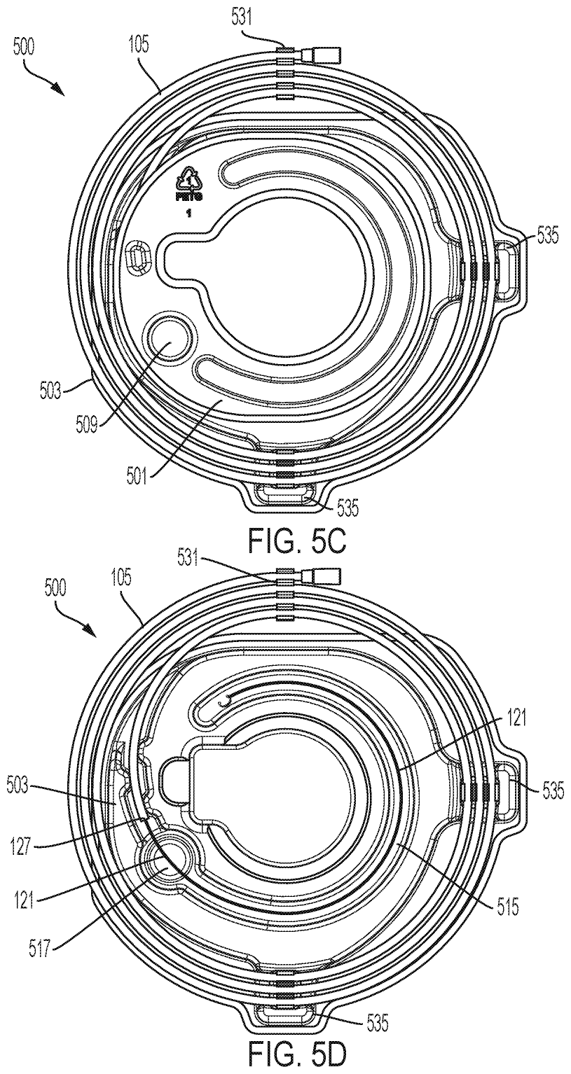

FIG. 5A depicts a top perspective view of another medical device package 500 including a lid portion 501 mated to a tray portion 503 with an outer sheath 105 and medical device assembly 107 disposed therebetween. FIG. 5B depicts a perspective exploded view of the medical device package 500, FIG. 5C depicts a top view of the lid portion 501, and FIG. 5D illustrates a top view of the tray portion 503 with the outer sheath 505 and the medical device assembly 507 disposed thereon. In various embodiments, the package 500 can include some or all of the features of the medical device packages 100 or 400 described elsewhere herein.

Referring to FIGS. 5A-5D together, the lid portion 501 can include a retention member 509 similar to the retention member 109 described elsewhere herein. For example, the retention member 509 can take the form of an indentation or depression in the body of the lid portion 501 and can be configured such that, when depressed by a clinician, the retention member 509 protrudes into contact with a corresponding engagement surface 517 of the tray portion 503. While holding the catheter 121 in place via the retention member 109, the clinician may manipulate the medical device 125 to sheath or unsheath it as desired.

In the illustrated example, the outer sheath 105 can be held together in a spiral configuration using clips 531. By using such clips 531, the tray portion 503 need not include a recess corresponding to the entire length of the outer sheath 105, but rather the recess 511 is dimensioned only to receive a distal portion of the outer sheath 105. When mated together, the outer sheath 105 can surround a raised central region 531 in which the recess 511 and the enlarged region 515 are formed. When the lid portion 501 is mated to the tray portion 503, the lid portion 501 can cover or enclose the recess 511, including the enlarged region 515 in which the distal portion of the medical device assembly 107 can be disposed. The outer sheath 105 can additionally be coupled to the tray portion 503 via receptacles 535 that are configured to mate with the clips 531, e.g., using a snap-fit, interference fit, or other mating technique.

### IV. CONCLUSION

Although many of the embodiments are described above with respect to systems and methods related to medical device packaging, the technology is applicable to other applications and/or other approaches. Moreover, other embodiments in addition to those described herein are within the scope of the technology. Although particular features of medical device packages are described above with respect to the illustrated examples, in various embodiments some or all of the features of the various example packages can be combined, substituted, and/or modified as desired. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-5D.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

Unless otherwise indicated, all numbers expressing dimensions, percentages, or other numerical values used in the specification and claims, are to be understood as being modified in all instances by the term “about.” Accordingly, unless indicated to the contrary, the numerical parameters set forth in the specification and attached claims are approximations that may vary depending upon the desired properties sought to be obtained by the present technology. At the very least, and not as an attempt to limit the application of the doctrine of equivalents to the scope of the claims, each numerical parameter should at least be construed in light of the number of reported significant digits and by applying ordinary rounding techniques. Additionally, all ranges disclosed herein are to be understood to encompass any and all subranges subsumed therein. For example, a range of “1 to 10” includes any and all subranges between (and including) the minimum value of 1 and the maximum value of 10, i.e., any and all subranges having a minimum value of equal to or greater than 1 and a maximum value of equal to or less than 10, e.g., 5.5 to 10.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A medical device package comprising:
a tray defining a recess configured to receive an elongated medical device therein;
a lid configured to cover at least a portion of the recess and retain the elongated medical device therein; and
a retention aperture formed in the lid, the aperture configured to provide a clinician with access to a medical device disposed within the recess to releasably retain the elongated medical device with respect to the tray.

2. The medical device package of claim 1, wherein the retention aperture has an area of between about 1 cm² to about 5 cm².

3. The medical device package of claim 1, wherein the retention aperture is configured to receive a clinician's thumb or finger therethrough.

4. The medical device package of claim 1, further comprising a medical device disposed within the recess, the elongated medical device comprising:
a catheter having a lumen;
a delivery wire extending through the catheter lumen; and
an interventional element coupled to a distal end portion of the delivery wire, wherein, in a sheathed state, the interventional element is disposed within the catheter lumen, and in an unsheathed state the interventional element is disposed distal to a distal end of the catheter lumen.

5. The medical device package of claim 4, wherein the interventional element is configured to be transitioned from the unsheathed state to the sheathed state by applying pressure to the catheter through the retention aperture to retain the catheter in position with respect to the tray, and then proximally retracting the interventional element with respect to the catheter by pulling the delivery wire.

6. The medical device package of claim 4, wherein the interventional element is configured to be transitioned from the sheathed state to the unsheathed state by applying pressure to the catheter through the retention aperture to retain the catheter in position with respect to the tray, and then distally advancing the interventional element with respect to the catheter by pushing the delivery wire.

7. The medical device package of claim 1, wherein the recess comprises a lumen of an outer sheath.

8. The medical device package of claim 1, wherein the recess defines a spiral channel having a proximal aperture at a radially outermost portion of the tray.

9. The medical device package of claim 1, wherein the recess is configured to receive the elongated medical device therein in a coiled state.

10. The medical device package of claim 1, wherein a distal end portion of the recess comprises an enlarged region configured to receive a distal end portion of the elongated medical device therein.

11. A medical device package comprising:
a tray defining a recess configured to receive an elongated medical device therein;
a lid configured to cover at least a portion of the recess and retain the elongated medical device therein; and
a retention member formed in the lid, the retention member configured to be releasably depressed into contact with a medical device disposed within the recess.

12. The medical device package of claim 11, wherein the retention member comprises a button.

13. The medical device package of claim 11, further comprising a medical device disposed within the recess, the elongated medical device comprising:
a catheter having a lumen;
a delivery wire extending through the catheter lumen; and
an interventional element coupled to a distal end portion of the delivery wire, wherein, in a sheathed state, the interventional element is disposed within the catheter lumen, and in an unsheathed state the interventional element is disposed distal to a distal end of the catheter lumen.

14. The medical device package of claim 13, wherein the interventional element is configured to be transitioned from the unsheathed state to the sheathed state by depressing the retention member to retain the catheter in position with respect to the tray, and then proximally retracting the interventional element with respect to the catheter by pulling the delivery wire.

15. The medical device package of claim 13, wherein the interventional element is configured to be transitioned from the sheathed state to the unsheathed state by depressing the retention member to retain the catheter in position with respect to the tray, and then distally advancing the interventional element with respect to the catheter by pushing the delivery wire.

16. The medical device package of claim 11, wherein the recess comprises a lumen of a outer sheath.

17. The medical device package of claim 11, wherein the recess defines a spiral channel having a proximal aperture at a radially outermost portion of the tray.

18. The medical device package of claim 11, wherein the recess is configured to receive the elongated medical device therein in a coiled state.

19. The medical device package of claim 11, wherein a distal end portion of the recess comprises an enlarged region configured to receive a distal end portion of the elongated medical device therein.

20. A method comprising:
providing a medical device package assembly comprising:
a tray defining a recess;
an elongated medical device disposed within the recess, the elongated medical device comprising a catheter and an interventional element;
a lid configured to cover at least a portion of the recess; and
a retention aperture formed in the lid;
contacting the catheter through the retention aperture;
manipulating the interventional element while contacting the catheter, thereby moving the interventional element proximally or distally with respect to the catheter.

21. The method of claim 20, wherein manipulating the interventional element comprises distally advancing or proximally retracting a delivery member coupled to the interventional element.

22. The method of claim 20, wherein contacting the catheter comprises pressing a thumb or finger onto the catheter through the retention aperture.
